(19) United States
(12) Patent Application Publication
(10) Pub. No.: US 2022/0218002 A1
(43) Pub. Date: Jul. 14, 2022

(54) METHOD FOR PROCESSING FEED GRAIN FOR DAIRY ANIMALS

(71) Applicant: Mark D. Holt, Phoenix, AZ (US)

(72) Inventor: Mark D. Holt, Phoenix, AZ (US)

(21) Appl. No.: 17/650,242

(22) Filed: Feb. 7, 2022

Related U.S. Application Data
(63) Continuation of application No. 16/407,576, filed on May 9, 2019, now abandoned.
Application No. 16/407,576 is a continuation of application No. 15/269,747, filed on Sep. 19, 2016, now abandoned.
Application No. 15/269,747 is a continuation of application No. 13/732,210, filed on Dec. 31, 2012, now Pat. No. 9,446,094.
(60) Provisional application No. 62/377,389, filed on Aug. 19, 2016.
(60) Provisional application No. 61/582,347, filed on Dec. 31, 2011.

(51) Int. Cl. A23K 50/10; A23K 20/163; A23K 20/147; A23K 40/20; A61K 31/718; A23K 40/25; A61K 9/00

(52) U.S. Cl. CPC A23K 50/10; A23K 20/163; A23K 20/147; A61K 9/0056; A61K 31/718; A23K 40/25; A23K 40/20

(57) ABSTRACT

Method of producing a feedstuff for a dairy animal that potentiates milk production. The method includes a multi-stage process having one stage in which a feed grain for dairy animals is heat-treated for a period of time at a temperature above 90 degrees Celsius. The grain is processed for another period of time that includes disrupting the prolamin/protein bonds which produces a hydrophilic, vitreous feedstuff having a starch and protein matrix composed at least partially by prolamin. The feed grain is heat-treated for a first period of time, of which at least 200 seconds is maintained above 90 degrees Celsius, and thereafter the feed grain is processed in a second stage by applying sufficient processing to disrupt the prolamin/protein bonds and thereby producing a hydrophilic, vitreous, feedstuff comprising a starch and protein matrix composed of at least three percent prolamin.

### CROSS REFERENCE TO RELATED APPLICATIONS

This application claims the benefit under 35 U.S.C. § 119(e) of U.S. Utility patent application Ser. No. 15/269,747, filed on Sep. 19, 2016, which claims benefit to U.S. Provisional Patent Application No. 62/377,389, filed on Aug. 19, 2016, and which is incorporated herein by reference in its entirety and which also constitutes a portion of the present disclosure as part of this patent specification. U.S. Utility patent application Ser. No. 15/269,747 is also a continuation of U.S. Utility patent application Ser. No. 13/732,210, filed on Dec. 31, 2012, now issued as U.S. Utility Pat. No. 9,446,094, which claims the benefit under 35 U.S.C. § 119(e) of U.S. Provisional Patent Application No. 61/582,347, filed on Dec. 31, 2011, each of which is incorporated herein by reference in its entirety and which also constitutes a portion of the present disclosure as part of this patent's specification.

### BACKGROUND

In the realm of animal digestion, the ruminant animal is one of the most diverse mammals in the world. Whereas, most mammals contain one stomach, the ruminant has four stomachs and a small intestine with a vast array of organisms with one of the most robust microbiological ecologies on the planet. In these four stomachs, it has been hypothesized that as many as 150,000 organisms reside and are prepared to digest nutritional substrates in vast quantities. The rumen ecology allows them to metamorphose bacteria that distinctly digest the substrate presentation.

Ruminants primarily digest carbon sources in the form of proteins, carbohydrates, fats, sugars and fiber. Ruminants are also unique in that the rumen ecology can ebb and flow regarding digestion and substrates and the ecology based on the substrate presentation. For example, a high corn diet will have a different ratio of bacteria, fungi, volatile fatty acids and protozoa than a high fiber diet.

Operating pH for the ruminant can range from 5.5-6.0, and up to 8.0 with the former representing a high starch/sugar diet and the latter being more fiber forage based. The rumen breaks these carbohydrates and sugars down into volatile fatty acids (VFA) in the form of acetic, lactic, propionic and butyric acid. Once they are broken down they are absorbed through the rumen wall and into the bloodstream.

Long chain fats are biohydrogenated in the rumen and absorbed in the small intestine. Crude protein substrates are hydrolyzed to peptides (chains of amino acids) and deaminated to ammonia. In a dairy animal, we find that most substrates that contribute to milk production are digested in the rumen and not post-ruminal. In a beef animal, primary concern is with digestion across the digestive tract. Post ruminal VFA's contribute less than 5% of the production of a dairy animal.

Further complicating digestion by the dairy animal are the bacteria, fungi and protozoa of the rumen that contribute up to 60% of the rumen mass. Most literature and models address the production of bacteria and fungi which have a specific passage rate and a lower level of amino acid contribution. Protozoa have a 6% per hour rumen over rate and contribute up to two times higher levels of essential amino acids such as lysine and methionine.

In contrast, most monogastric animals (i.e. pigs and chickens) have a pH site digestion in the 2-4 pH range. This allows monogastrics much greater flexibility with regard to substrate digestion, starch hardness and biological efficiencies for meat production. Further confounding the digestion efficiency of dairy animals is the current practices aimed at increasing corn production that have increased characteristics of the corn that are detrimental to the efficiencies of lactating dairy animals, and particularly dairy cows. These complexities of prolamins, particularly zein in corn can reduce the efficiency of the digestion of corn up to 60-80% in the rumen and therefore reduce milk production significantly.

### BRIEF DESCRIPTION OF THE DRAWINGS

In the accompanying drawings:

FIG. 1 graphically depicts certain percentage based measurements taken at 0, 2, 4, and 6 hours of yellow corn meal Reference Human Food Grade NDF digests of rumen digestion residue samples;

FIG. 2 depicts similar measurements, but for white whole corn, course ground before processing;

FIG. 3 depicts similar measurements, but for ground corn, fine grind before processing;

FIG. 4 depicts similar measurements, but for pooled white corn before processing;

FIG. 5 depicts similar measurements, but for low gelatinization flaked corn;

FIG. 6 depicts similar measurements, but for high gelatinization flaked corn;

FIG. 7 depicts similar measurements, but for pooled flaked corn;

FIG. 8 depicts similar measurements, but for extruded corn, before fine grinding, course ground for study;

FIG. 9 depicts similar measurements, but for processed corn fine grind;

FIG. 10 depicts similar measurements, but for pooled processed corn;

FIG. 11 depicts cell counts and microflora at 2 and 4 hours for variously processed corn;

FIG. 12 illustrates a digestive tract of a ruminant depicted as a dairy cow;

FIG. 13 illustrates the hydrophilic characteristic of Rumen Available Starch (RAS) produced according to the teachings of the present disclosure on the left compared to the hydrophobic characteristic of ground corn on the right, considering the same amount of water in the two glasses;

FIG. 14 illustrates and characterizes formulaically RAS percent starch digestibility, in-vitro, over a 24 hour period;

FIG. 15 illustrates and characterizes formulaically high gelatinization flaked corn percent starch digestibility in the rumen over a 24 hour period;

FIG. 16 illustrates and characterizes formulaically percent starch digestibility in the rumen over a 24 hour period for course ground white whole corn, pre-extrusion processing;

FIG. 17 illustrates and characterizes formulaically percent starch digestibility in the rumen over a 24 hour period for RAS processed, fine ground corn;

FIG. 18 illustrates and characterizes formulaically percent starch digestibility in the rumen over a 24 hour period for RAS processed, course ground corn;

FIG. 19 tabulates pregnancy rates, on a monthly basis, comparing RAS treated cows in the right-most column versus control group cows in the center column that did not receive RAS feed and which demonstrates increased pregnancy rates of 18.4% in the hot, summer month of July; 125.3% in the hot, summer month of August; and 76.8% in the hot, summer month of September;

FIG. 20 illustrates starch source effects on culture pH by day of incubation;

FIG. 21 illustrates starch source effects on pH by time after feeding on day 10;

FIG. 22 details two analyses of the composition of degermed corn and RAS processed corn;

FIG. 23 tabulates percent digestion of RAS in a ruminant's rumen at 2 hours (40.6%), 4 hours (61.9%), 6 hours (78.3%), 12 hours (85.2%) and 24 hours (94.0%); and

FIG. 24 tabulates a comparison between 1000 grams of RAS versus 1000 grams of corn regarding grams of starch (900 grams vs. 720 grams); percent rumen digested at 7 hours (85% vs. 18%) and grams of rumen available starch (765 grams vs. 130 grams).

### DESCRIPTION

In at least one embodiment, a method is disclosed for processing a prolamin-containing feed source into a gelatinous feedstuff. The feedstuff is fed to ruminant animals for the purpose of potentiating either milk production or conception, or both. In at least one example, the feed source is corn and the ruminant animal is a bovine, and more specifically, a cow. In one particular example, the animal is a milking cow and the pH of its relevant digestive environment is in the range of 5.5 to 8.0. The method includes processing, by extrusion, a prolamin-containing feed source comprising (including, but not limited to) a starch-protein matrix within which the included protein is composed of three percent or greater prolamin. This processing produces a hydrophilic gelatinous feedstuff that has starch and protein content. The hydrophilic gelatinous feedstuff is fed to a ruminant animal. A rumen-retained portion of the fed feedstuff is retained within the rumen of the animal for at least a twenty-four hour period, and during the first twenty-four hours of that period, at least seventy-five percent and up to ninety-nine percent of the starch content of the rumen-retained portion of the fed feedstuff is digested. The period of retention may be shorter in the instance of fast-transit, high-digestibility starch matrices.

The affects and benefits of feeding this unique feedstuff to dairy cows surprisingly includes an increase in rumen pH. The effects of this are substantial as grain starch, such as in corn, normally has required buffering to prevent the death of bacteria and Protozoa. This increased pH effect is responsible for an increase in Neutral Detergent Fiber (NDF) digestibility. Heretofore, when starch has been added, NDF digestibility has gone down due to the shift or decrease in pH, which is opposite to the described experience of increased pH as a result of feeding the currently disclosed feedstuff to ruminant animals.

Additionally, there is a stratification effect in the rumen relative to the processed feedstuff. The presently disclosed feedstuff is lighter than corn, but more specifically is sufficiently light (low density) to float to the top of the rumen where the protozoa reside causing creep feeding of the bacteria and Protozoa. These growth rates are two to three times greater than for corn starch.

Regarding the Protozoan effect described immediately above, the presently disclosed feedstuff grows Protozoa which are 22% lysine. Based on these turnover rates, the results of feeding the feedstuff exceed all protein requirements of a high producing dairy cow without the direct addition of any protein to the diet.

Regarding the passage rate of the presently disclosed feedstuff, ground corn and other typical forms of corn pass through the rumen at a rate of 10-25% per hour. In contrast, the presently disclosed feedstuff, given its post-processing characteristics, does not leave the rumen until substantially fully digested, which corresponds to the surprisingly decreased rate of passage from the rumen of 0.5-1.0% per hour.

Regarding beef cattle production and efficiency, the presently disclosed feedstuff increases pH which reduces acidosis. It also increases Microbial Bacteria (MB) and Microbial Protozoa (MP) and increases the rate of gain due to enhanced delivery of amino acid.

Among other benefits, the need to feed sodium bicarbonate and yeast is reduced or eliminated by the feeding of the presently disclosed feedstuff that has been accordingly processed. The need to add a separate protein is also reduced or eliminated due to the increase endogen. Paramount, the amount of grain (corn) fed to the animal will be drastically reduced while at the same time delivering the same amount of nutrient to the animal.

Corn starch and the presently disclosed feedstuff have been compared. A comparison fermentation has been performed in a shaking incubator using mixed ruminal microbes from lactating cows. In the fermentation tubes, a change was observed in the mass of the microbial brown and green material (the microbial mass did not stain with ruthenium red which would stain carbohydrates, apparently mixed composition carbohydrates, but also starch) in the presently disclosed feedstuff versus corn starch (CS; both added at 0.15 g/tube, fermented with a lower N Goering and Van Soest medium for 0, 1, 2, and 4 h). The microbial masses started out similarly at 0 hours. The mass in the tube with the presently disclosed feedstuff was substantially greater than that mixed with corn starch as time increased. Qualitatively, it looked like the protozoa contained more starch granules in the corn starch fermentations, but had more amorphous material in the form of microbial mass with the presently disclosed feedstuff.

The starch granules in the presently disclosed feedstuff fermentation seemed more clumped than those in the corn starch sample, indicative of the fact that the physical form of the presently disclosed feedstuff creep feeds the bacteria as the physical size reduces ingestion of the presently disclosed feedstuff by protozoa.

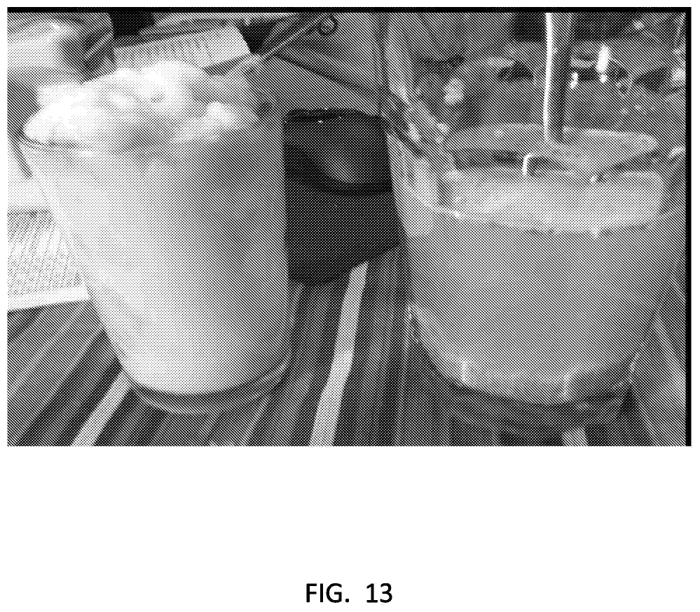

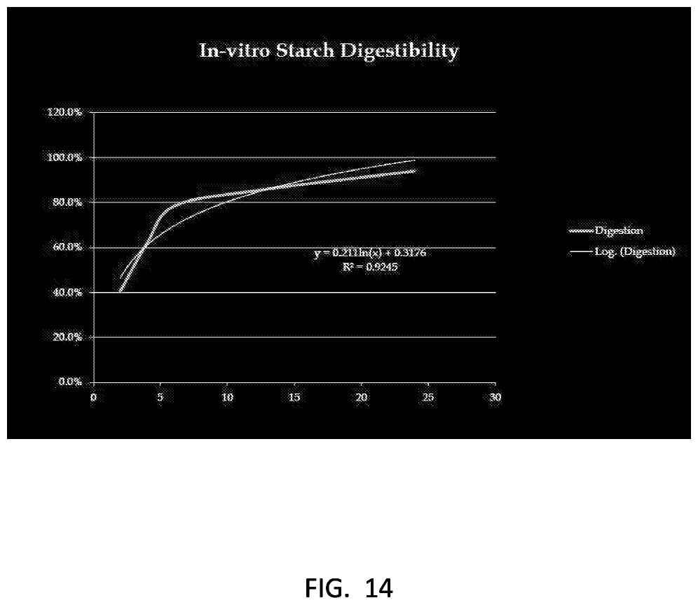

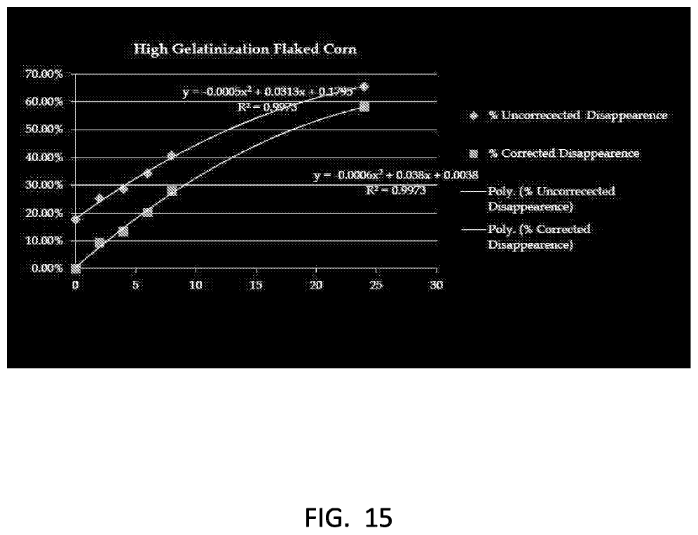

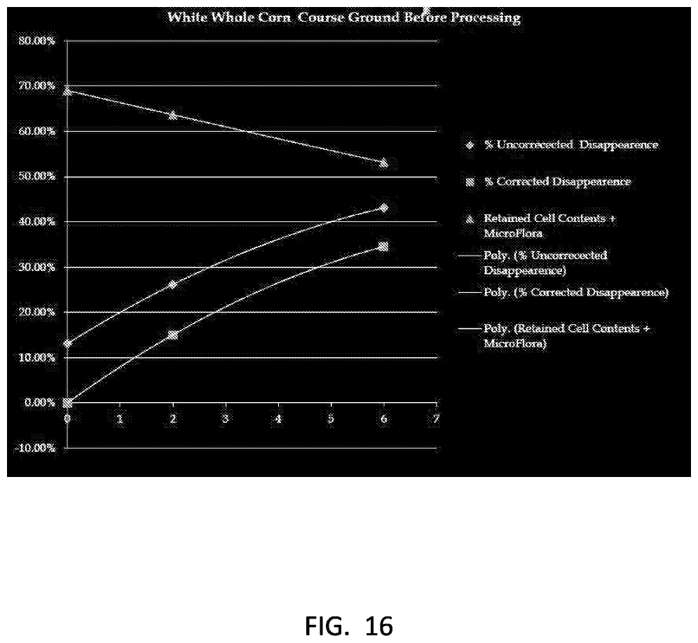

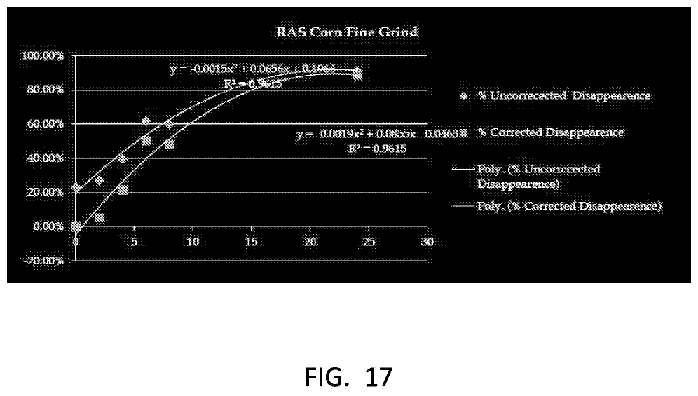

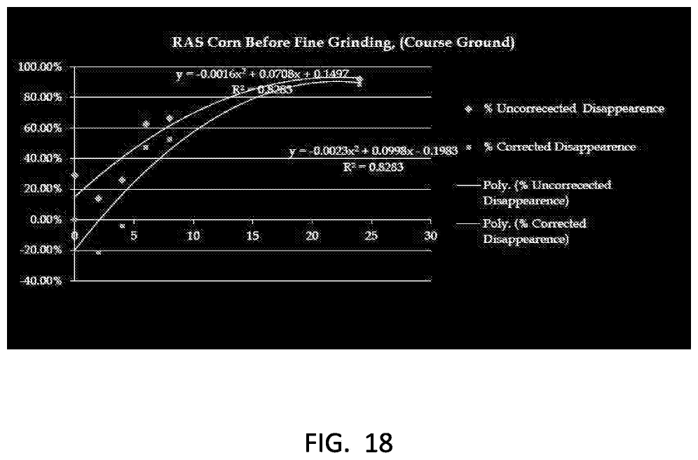

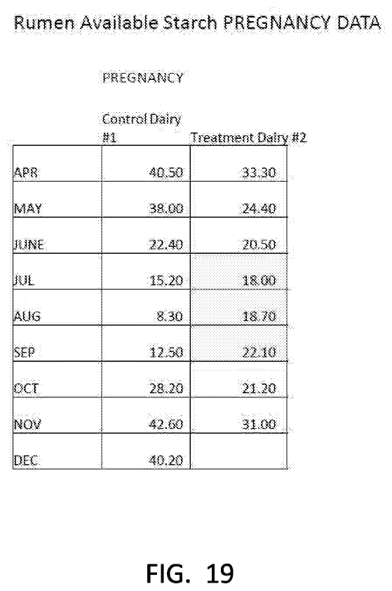

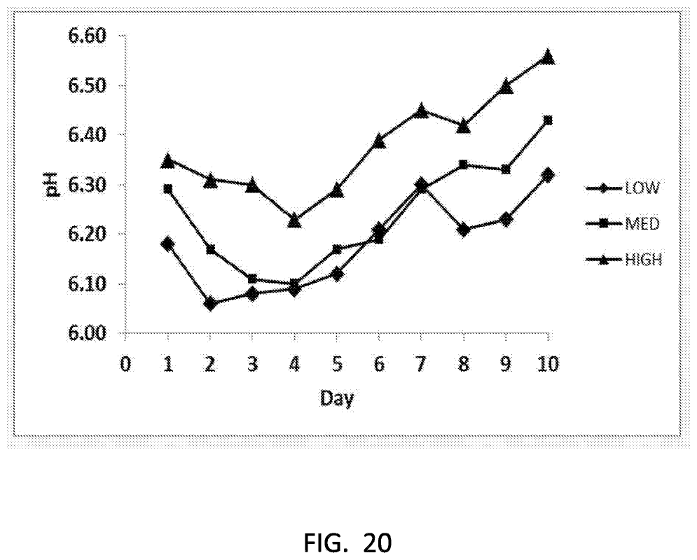

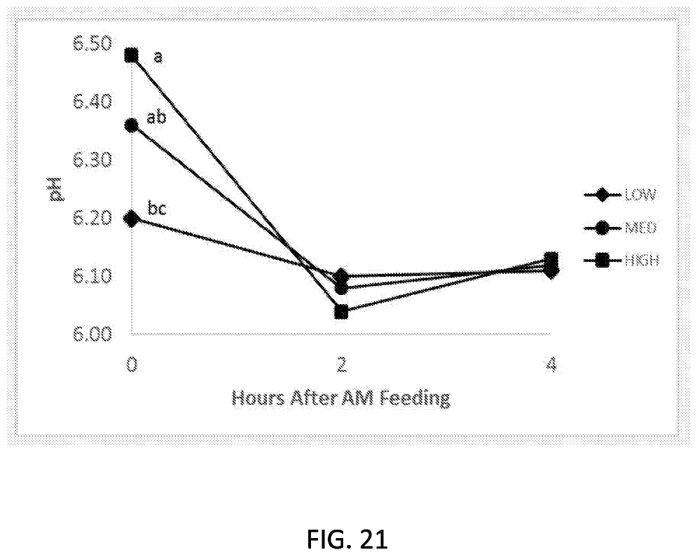

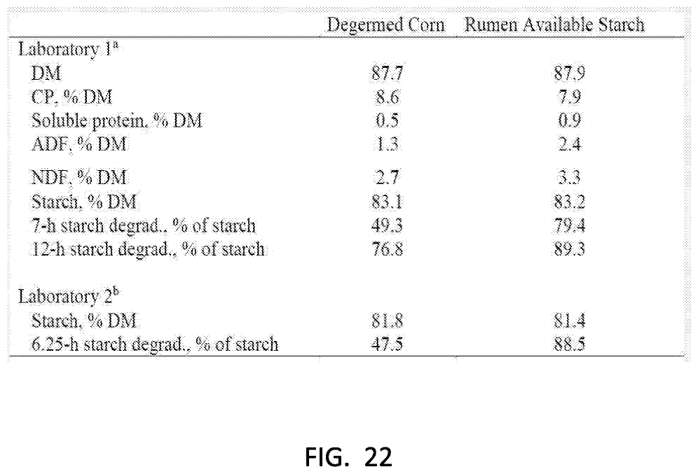

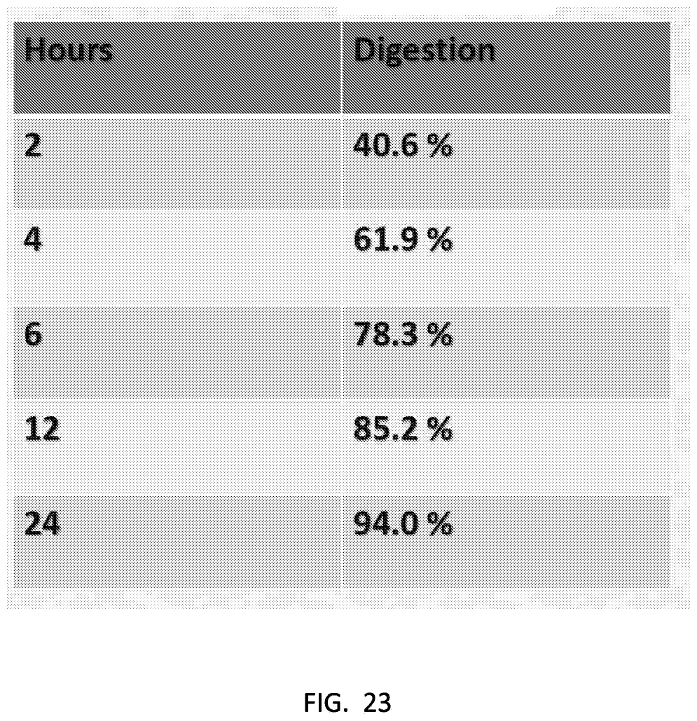

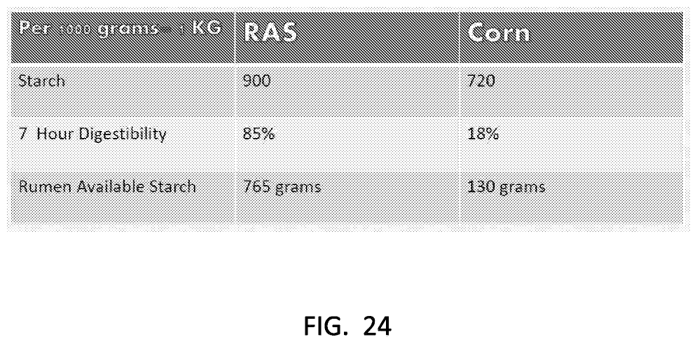

Interestingly, the microbes observed appear to be Streptococcus. Some starch utilizers were attached to the starch granules, but surprisingly, as is depicted in the accompanying photograph of FIG. 13, there are also masses of chains of microbes that are not attached.

As a functionality, the presently disclosed feedstuff is pushing unsaturated fatty acids out of the rumen which lessens their antimicrobial impact on fermentation. Furthermore, it delivers more essential fatty acids post-ruminally for weight gain and reproduction performance increases, was well as increasing immune function, milk production and overall health of the animal.

Regarding protein delivery, it is observed that the Protozoa engulfs itself in soluble starch, increases specific gravity and falls out of the top portion of the rumen material to flow out of the rumen, which has been test tube observed.

In the present disclosure, the terminology “gelatinous” is defined as non-vitreousness or lowered-vitreousness which indicates an enhanced porosity that enables the rumen fluid bacteria, protozoa and fungi to have greater access to degrade the feedstuff. At least in part, vitreous properties in this disclosure are defined as 2,000 centipoise or less.

In the present disclosure, the terminology “extrusion” defines a process that includes the application of pressure, thermal, mechanical and/or chemical shear (PTMCS) or combination thereof to the feed source to disrupt the prolamin/protein bonds.

Prolamins are a group of plant storage proteins having a high proline content and are found in the seeds of certain cereal grains including wheat (gliadin), barley (hordein), rye (secalin), corn (zein), sorghum (kafirin) and as a minor protein, as avenin in oats, and each of which cereal grains can serve as the base or original grain to be processed into the presently disclosed feedstuff. They are characterized by a high glutamine and proline content and are generally soluble only in strong alcohol solutions.

In at least one embodiment, the digested percentage of the starch content of the rumen-retained portion of the fed feedstuff during the first twenty-four hours of the at least twenty-four hour period is at least eighty percent.

In another embodiment, the digested percentage of the starch content of the rumen-retained portion of the fed feedstuff during the first twenty-four hours of the at least twenty-four hour period is at least eighty-five percent.

In another embodiment, the digested percentage of the starch content of the rumen-retained portion of the fed feedstuff during the first twenty-four hours of the at least twenty-four hour period is at least ninety percent.

In another embodiment, the digested percentage of the starch content of the rumen-retained portion of the fed feedstuff during the first twenty-four hours of the at least twenty-four hour period is at least ninety-four percent.

In another embodiment, the digested percentage of the starch content of the rumen-retained portion of the fed feedstuff during the first twenty-four hours of the at least twenty-four hour period is at least ninety-five percent.

In another embodiment, the digested percentage of the starch content of the rumen-retained portion of the fed feedstuff during the first twenty-four hours of the at least twenty-four hour period is at least ninety-eight percent.

In another embodiment, the digested percentage of the starch content of the rumen-retained portion of the fed feedstuff during the first twenty-four hours of the at least twenty-four hour period is at least ninety-eight and six-tenths percent.

In another embodiment, the digested percentage of the starch content of the rumen-retained portion of the fed feedstuff during the first two hours of the at least twenty-four hour period is at least thirty percent.

In another embodiment, the digested percentage of the starch content of the rumen-retained portion of the fed feedstuff during the first two hours of the at least twenty-four hour period is at least thirty-seven percent.

In another embodiment, the digested percentage of the starch content of the rumen-retained portion of the fed feedstuff during the first two hours of the at least twenty-four hour period is at least thirty-seven and three-tenths percent.

In another embodiment, the digested percentage of the starch content of the rumen-retained portion of the fed feedstuff during the first two hours of the at least twenty-four hour period is at least forty percent.

In another embodiment, the digested percentage of the starch content of the rumen-retained portion of the fed feedstuff during the first two hours of the at least twenty-four hour period is at least forty and six-tenths percent.

In another embodiment, the digested percentage of the starch content of the rumen-retained portion of the fed feedstuff during the first six hours of the at least twenty-four hour period is at least seventy percent.

In another embodiment, the digested percentage of the starch content of the rumen-retained portion of the fed feedstuff during the first six hours of the at least twenty-four hour period is at least seventy-eight and three-tenths percent.

In another embodiment, the digested percentage of the starch content of the rumen-retained portion of the fed feedstuff during the first seven hours of the at least twenty-four hour period is at least eighty percent.

In another embodiment, the digested percentage of the starch content of the rumen-retained portion of the fed feedstuff during the first seven hours of the at least twenty-four hour period is at least ninety-one and four-tenths percent.

In another embodiment, the digested percentage of the starch content of the rumen-retained portion of the fed feedstuff during the first twelve hours of the at least twenty-four hour period is at least seventy-five percent.

In another embodiment, the digested percentage of the starch content of the rumen-retained portion of the fed feedstuff during the first twelve hours of the at least twenty-four hour period is at least eighty-five and two-tenths percent.

In another embodiment, the extrusion process ruptures the prolamin bonds thereby rendering at least ninety-eight percent of the starch content of the hydrophilic resulting gelatinous feedstuff digestible in the rumen of ruminant animals, which is also referred to as Rumen Available Starch (RAS) which is similarly highly digestible.

In another embodiment, a viscosity of the hydrophilic resulting gelatinous feedstuff is less than 2000 Centipoise. In another aspect, the hydrophilic resulting gelatinous feedstuff can be characterized as possessing less than 50% of the viscosity value of the corresponding non-treated substrate.

In another embodiment, conception rates are increased in a ruminant animal fed the hydrophilic gelatinous feedstuff during periods of potential heat stress when ambient temperatures daily exceed ninety degrees Fahrenheit.

In another embodiment, the likelihood of conception of a ruminant animal fed the hydrophilic gelatinous feedstuff is at least doubled during periods of heat stress when ambient temperatures daily exceed one-hundred degrees Fahrenheit.

In another aspect, the presently disclosed processing of the feed source increases the uptake of starch by Entodinia protozoa, propagation of the latter, and increases the output to the small intestine of high quality essential amino acids to the small intestine (i.e. lysine, methionine, cysteine).

A production method and resulting feedstuff composition for ruminant animals is disclosed that includes: the manufacture of feed from grains containing prolamine; the manufacture of feed with a defined characteristic from high prolamine grains; a method for rupturing the starch prolamine matrix for a feedstuff; and a method of preparing a feed to improve the performance and/or feed utilization by microorganisms and/or animals.

Animal feeding operations house groups of animals that are fed together with the goal of maximizing their growth, while minimizing their food intake. Typically, the feed is generally composed of starch and protein containing substances. In many cases, corn, processed corn or by-products of corn processes and fermentations of corn are fed to animals. Similarly, the original starch/protein source can be sorghum or another type of grain. Corn is typically favored for its relatively high nutrient and starch composition, as well as low cost.

The availability of the starch, protein and other nutrients from the grains can be improved by increasing the surface area of the feedstuff by grinding, milling and flaking the original material. The overall digestibility of the grain, and corn in particular, can also be improved by applying heat and/or heat under pressure. Still further, digestability of corn can also be improved by hydrating it.

Lactating dairy cows have four stomachs for the digestion of food; however, the rumen is the most important for milk production as it is the area where most components (i.e. starch, protein and fiber) are broken down for milk production. It has been concluded through research that 80-95% of the starch broken down in the rumen contributes to Volatile Fatty Acid (VFA) production, and which is further broken down into milk production. Moreover, properly processed starch that is available in the rumen can also contribute to the production of microbial protein that is the primary source of protein produced by the cow for milk synthesis.

Most conventional starch sources fed to dairy cattle are moderately processed through steam heating and flaking and produce products in which only one-third of the starch is degraded in the rumen. Further, in the last 30 years of corn breeding, the characteristics of corn have been selected to produce grain that is: harder, higher in zein proteins (a protein matrixed with starch that reduces digestibility to dairy cattle), higher in test weights, faster to dry, has fewer fines, is more hydrophobic instead of hydrophilic, and is higher yielding. Each of these characteristics are detrimental to the digestion of starch in the rumen which breaks the starch down at a pH of 6.0-7.5, as opposed to a monogastric (i.e. chicken, pig, layer, turkey) that can digest these new corn varieties because the pH in their digestive tracts can be as low as 2-3. Further, the digestion sites in the monogastric, as well as a production beef cow, is across the total digestive tract and does not necessarily have to be site specific in the breakdown of starch and protein to optimize the animals' production.

In ruminants, the most efficient manner to produce milk with the currently available substrates is to make the starch fraction more available in the rumen in a form that is vitreous enough to adhere to the particles in the rumen and can nearly completely degrade before leaving the rumen. This yields the most efficient use of starch sources and increases the production of protein in the rumen, thereby sparing the waste of starch sources and increasing the endogenous production of protein to the dairy animal. This will increase milk production with the least amount of nutrients and reduce the excretion of excess nitrogen and the global footprint of dairy cows worldwide.

The present description discloses, among other things, a manufacturing process for cereal grains, as well as the feedstuff that results therefrom and which provides a highly digestible starch source by changing the properties of the zein/starch matrix to be hydrophilic from hydrophobic. Minimally, this is accomplished by processing a ground starch source (whole kernel corn, ground corn, sorghum, wheat, rye or other grain, for example) in a pressure vessel at between 7-40 psi and a temperature of 200-325 degrees Fahrenheit for a time period of one-half to five minutes in dependence upon the starch/zein matrix of the starch source. Further processing includes extrusion in which mechanical pressure and shear is applied until the matrix has been gelatinized to between approximately 70-100 percent. The resulting product has vitreous properties that enhance the digestion of the original starch source by the fed animal.

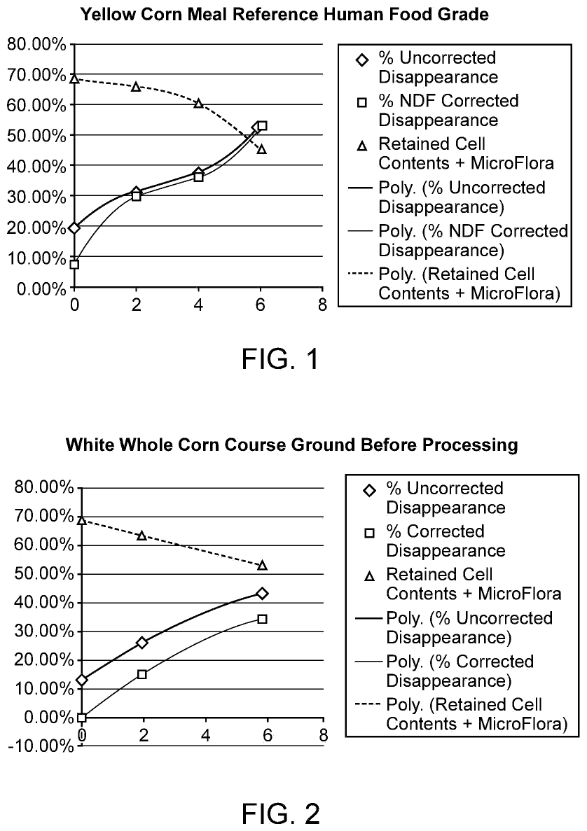

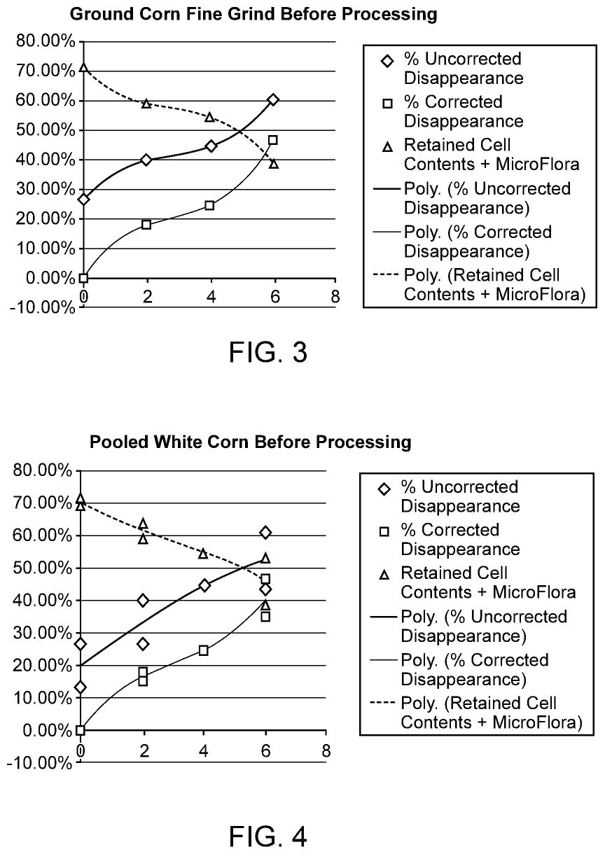

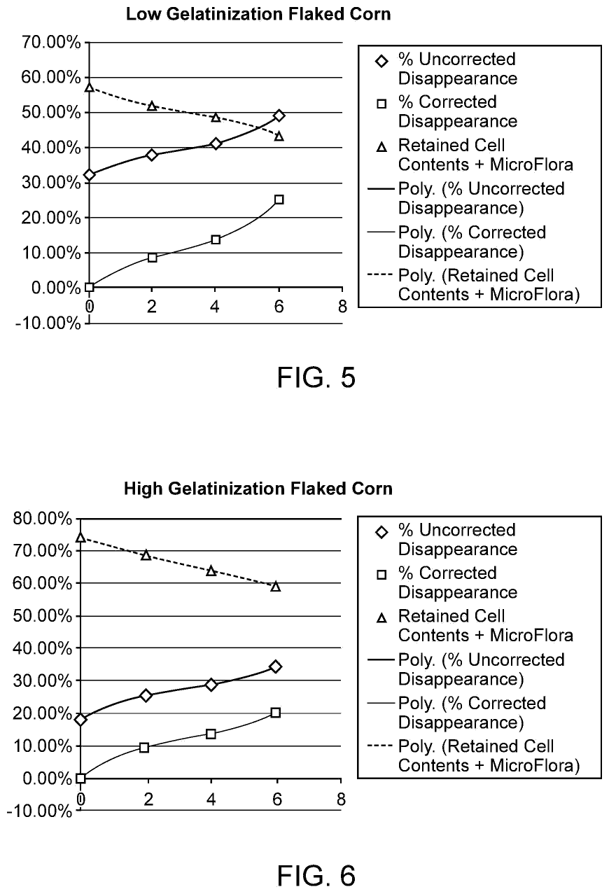

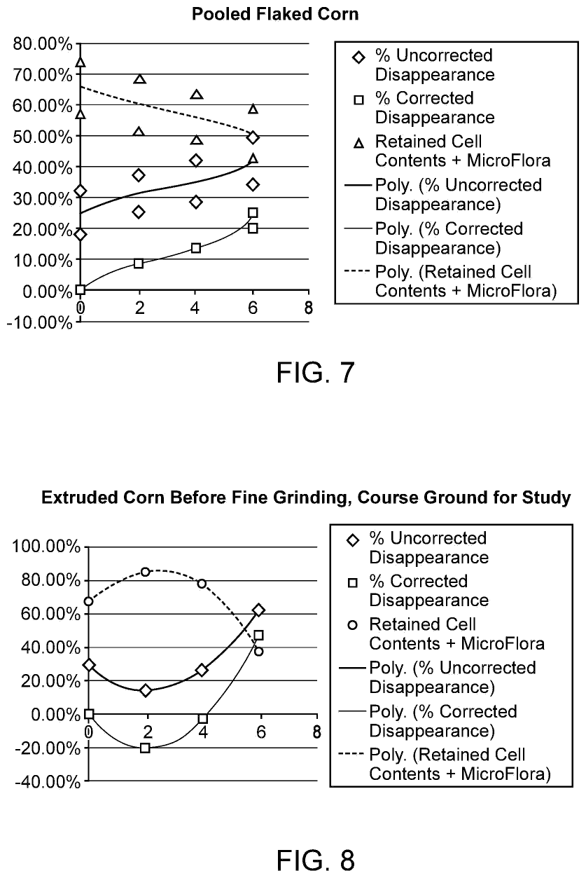

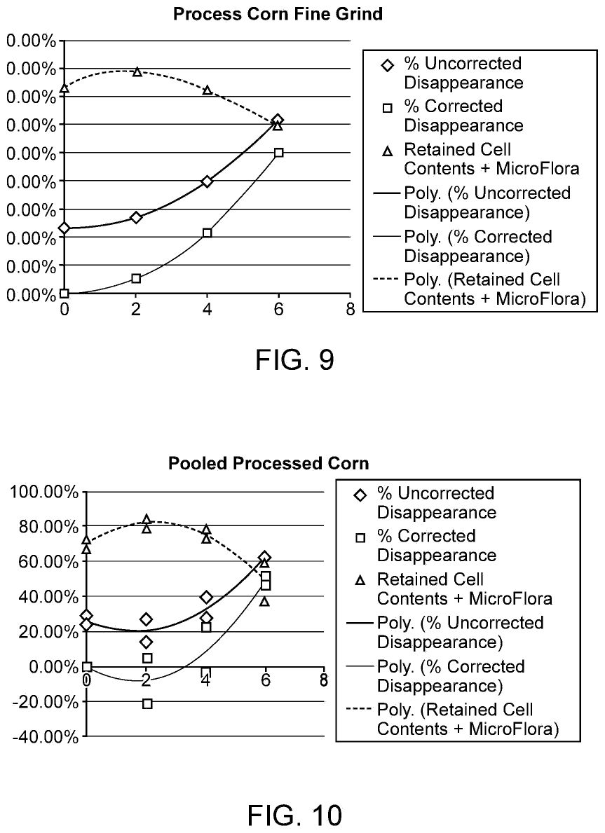

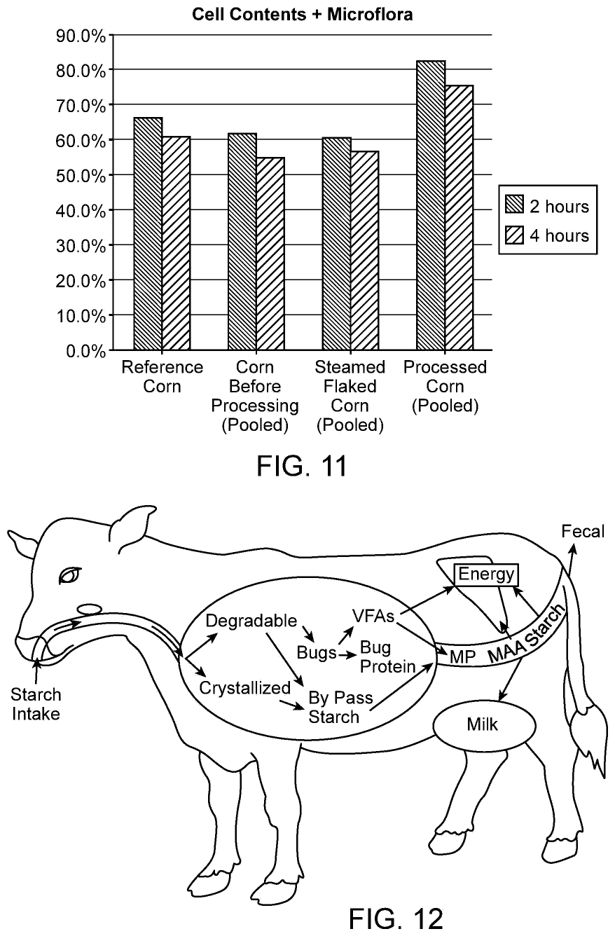

As an example, an end product is created that has vitreous properties that enhance the absorption of rumen fluid and bacteria which enhance the breakdown of starch within the rumen. The breakdown of this starch is found to be an inverted parabola digestion curve as opposed to a curvilinear digestion curve which means that the degradation rate initially drops versus conventional starch, and then degrades rapidly because of vitreous and hydrophilic properties enhanced by the production properties. These digestion curves and relationships are found in the accompanying FIGS. 1-10 which show graphical figures that compare the digestion curves of conventional starch sources of ground corn and flaked corn.

In these regards, a method of producing a feedstuff for a dairy animal is described that potentiates milk production. The method includes a multi-stage process comprising one stage in which a feed grain, of the types described herein, for dairy animals is heat-treated for a period of time at a temperature above 90 degrees Celsius and another stage in which the grain is processed for another period of time and which comprises disrupting the prolamin/protein bonds and thereby producing a hydrophilic, vitreous feedstuff comprising a starch and protein matrix composed at least partially by prolamin.

In a further aspect, the stage in which the feed grain is heat-treated for a period of time at a temperature above 90 degrees Celsius is for at least 100 seconds, but less than 1200 seconds, and all times in between.

In still a further aspect, the stage in which the feed grain is heat-treated for a period of time at a temperature above 90 degrees Celsius is at least 200 seconds, but less than 1200 seconds, and all times in between.

In a further aspect, the temperature at which the feed grain is heat-treated for a period of time at a temperature above 90 degrees Celsius is at least 100 degrees Celsius, but less than 500 degrees Celsius, and all temperatures in between.

In still a further aspect, the temperature at which the feed grain is heat-treated for a period of time at a temperature above 90 degrees Celsius is at least 150 degrees Celsius, but less than 500 degrees Celsius, and all temperatures in between.

In yet a further aspect, the temperature at which the feed grain is heat-treated for a period of time at a temperature above 90 degrees Celsius is at least 200 degrees Celsius, but less than 500 degrees Celsius, and all temperatures in between.

In a further aspect, the starch and protein matrix comprises at least three percent prolamin.

In still a further aspect, the starch and protein matrix comprises at least five percent prolamin.

In a further aspect, the method further comprises a first stage in which the feed grain is heat-treated for a first period of time, of which at least 200 seconds, but less than 500 seconds, and all times in between, is maintained above 90 degrees Celsius, but less than 500 degrees Celsius, and all temperatures in between, and thereafter the feed grain is processed in a second stage by applying sufficient temperature and/or pressure processing to disrupt the prolamin/protein bonds, thereby producing a hydrophilic, vitreous, feedstuff comprising a starch and protein matrix composed of at least three percent prolamin.

In still a further aspect, the method further comprises extruding the feed grain in a second stage by applying sufficient shear pressure to disrupt the prolamin/protein bonds, thereby producing an extruded hydrophilic, low-vitreous, gelatinous feedstuff comprising a starch and protein matrix composed at least partially of prolamin.

In a further aspect, the feed grain comprises at least one of wheat, barley, rye, corn, sorghum and oats.

In still a further aspect, the feed grain has a vitreousness of at least 66% prior to being heat-treated.

In another aspect, the method further comprises a first stage of the multi-stage process in which the feed grain is heat-treated for a period of at least 1200 seconds. Alternatively, the 1200 second long first stage of heat treatment can be less than or approximately 500 seconds, and all times in between.

In still another aspect, the method further comprises a first stage of the multi-stage process in which the feed grain is heat-treated for at least a 200 second period during which the heat treatment is maintained above 90 degrees Celsius, and that 200 second period is the last 200 seconds of the at least 1200 second long first stage of heat treatment. Alternatively, the 1200 second long first stage of heat treatment can be less than or approximately 500 seconds, and all times in between.

In another aspect of the present disclosure, a method of potentiating milk production in a dairy animal is described. The method comprises obtaining a hydrophilic, vitreous feedstuff that is manufactured by a multi-stage process comprising one stage in which a feed grain for dairy animals is heat-treated for a period of time at a temperature above 90 degrees Celsius. The grain is further processed using heat and/or pressure for another period of time and which comprises disrupting the prolamin/protein bonds, thereby producing the hydrophilic, vitreous feedstuff comprising a starch and protein matrix composed at least partially by prolamin. This method optionally further includes feeding the hydrophilic, vitreous feedstuff to a ruminant animal, and in which the fed feedstuff comprises a starch and protein matrix composed at least partially by prolamin.

In still another aspect, the method further comprises retaining a portion of the feedstuff within the rumen of the animal for at least a twenty-four hour period such that during the first twenty-four hours of that period, at least seventy-five percent of the starch content of the rumen-retained portion of the fed feedstuff is digested.

In yet another aspect, the method further comprises increasing the uptake of starch granules in Entodinia sp. rumen protozoa whereby higher propagation rates, lysis and delivery to the animal's small intestine of high quality essential amino acids is affected.

In another aspect, the method further comprises at least doubling the likelihood of conception of a ruminant animal fed the feedstuff during periods of heat stress when ambient temperatures exceed one-hundred degrees Fahrenheit.

Regarding the disclosed process feedstuffs, further modeling through the Cornell Nutrition Carbohydrate Protein System (CNCPS) has shown the process to increase degradation curves two to three times over conventional starch sources, and enhance the ability to contribute to protein synthesis thereby reducing the need for exogenous protein sources by 15-35% which also reduces nitrogen excretion by dairy cows.

Consistency in animal feed products, particularly for ruminant animals, and especially for beef cattle and dairy cows is highly desired and the resulting product of the process described above preferably has the following characteristics: consistent starch/protein/nutrient composition even with varied starting levels of prolamine and starch content; over 50% is digested within eight hours in the gastrointestinal tract; and comprises a stable chemical composition that resists degrading under storage conditions.

In ruminant animals, starch is variably processed: some is degraded in the rumen and grows bugs (bacteria); some makes VFA and microbial protein; some escapes the rumen before it can be degraded; some is utilized as an energy source; some goes to the large intestine before it can be absorbed; some grows bugs and provides VFA, but no protein to the cow; and some is indigestible by the animal and ends up in the manure.

The endosperm of corn is a starch protein matrix that comprises four types of protein: albumins, globulins, glutelins, and prolamines. Prolamines in corn are referred to as zein and make-up approximately 50-60% of the protein in corn. The amino-acid in prolamines makes the corn hydrophobic and therefore not soluble in water or rumen fluid. As such, prolamines have industrial applicability as a material for manufacture of such things as edible, biodegradable plastic.

Additional prolamine characteristics include that it forms on the starch granule surface; its proteins can cross-link; it encapsulates starch into a matrix; it advances with maturity like NDF in forages; and, it can have genetic differences in corn. Relatedly, floury/opaque corns are missing the Y-zein gene and are low in prolamines. Flint corns are very high in prolamines. Common corn hybrids are moderately-high in prolamines. For comparison, barley (hordein) and oats (avenin) are low in prolamines, wheat (gliadin) and rye (secalin) are med-low in prolamines, corn (zein) is high and sorghum (kafirin) is very high in prolamines.

Extruding corn is superior to rolling and flaking corn which does not physically change the starch/prolamine content of the corn. Further, as flakes of corn sit in inventory, the starch retrogrades in that it becomes more crystalline, which is indigestible and must be used within 2-3 days of production.

Extrusion as a processing method for corn is superior because it increases consistency. Extrusion also physically/chemically disrupts the starch/prolamine relationship and it does not retrograde. Extrusion increases shelf life, post processing, which increases the possibility that corn can be extruded at a central location and then shipped out. That is to say, extruded corn is “shelf stable”.

Exemplarily, corn can be comprised of as much as 89% dry matter, 9.1 percent crude protein, 9.9 NDF, 1.2% Lignin, 2.5% sugar, 70% starch and 88% total digestible nutrients.

Some of the benefits and characteristics of corn extruded according to this disclosure, and its utilization as a feed for ruminants, include: (1) decreased DMI (Dry Matter Intake); (2) increased milk yield; (3) changes in hepatic oxidation; (4) decreases heat stress; (5) decreases passage rate of starch; (6) increases microbial utilization of starch; (7) increases microbial protein production in the rumen; (8) increases microbial protein production in the intestine; (9) alters starch digestion curves; (10) increases the utilization of zein; (11) increases utilization of prolamine; (12) fosters site-specific (rumen/intestine/hindgut) digestion of the processed feed; (13) fosters digestion of the feed in the rumen by microorganisms; (14) increases surface area of the processed feed; (15) chemically modifies starch and protein composition; (16) decreases intestinal digestion of starch; (17) alters VFA profiles in the rumen; and (18) increases VFA production in the rumen and decreases VFA production in the hindgut.

Currently, tools exist that permit the expression of digestion kinetics in a manner that predicts field outcomes in the form of milk volume, fat and protein of the lactating dairy animal. Dietary carbohydrates are partitioned into A1-4 (sugar and organic acids), B1-2 (starch and soluble fiber), B3 (digestible fiber) and C (indigestible residue). The “B” portion of the carbohydrate fractions is of the utmost importance to the dairy animal as the level of starch and the location where it is digested is of utmost importance in order to feed the animal economically.

Starch can be completely digested in the rumen although many factors must be considered to foster 100%, or substantially 100% digestion. Digestion of starch and the subsequent breakdown and viscosity are key criteria to determine maximum efficiency of the milk producing animal. Primary degradation occurs from the breakdown by protozoa and microbial bacteria.

In addition, out flow of starch to the small intestine alters signals to decrease the Dry Matter Intake (DMI) to the cow. An increase in propionate to the liver has been found to decrease the DMI of the animal, thereby reducing the milk yield of the dairy animal. Therefore, from an efficiency perspective, the optimum site of the B1 pool for digestion by a dairy animal is in the rumen for milk production.

Optimal use of corn as a carbohydrate source to the rumen has been found to be based at least in part on chemical composition, processing techniques, surface area, final viscosities, and mechanical and thermal processing variables.

Increasing the rate and extent of starch fermentation in the rumen has been found to increase the levels of circulating propionate in the liver. Consequently, shifting starch digestion to the intestines, instead of the rumen, would theoretically provide more glucose to the animal, but at the expense of microbial growth which in turn should reduce protein efficiency in the animal. Importantly, conveying glucose sources to the small intestine does not increase glucose available for milk production.

It has been found, and it is presently disclosed that the utilization of RAS produced according to the present teachings, especially using heat and extrusion on whole or ground corn, increases starch digestion in the rumen 535% over ground corn retention in the rumen based on approximately 5,000 samples. This manufacturing process for RAS ruptures the Prolamin bonds making it possible for up to 98% of the RAS starch to be digested by rumen bacteria (bugs) within 24 hours of ingestion into the rumen. Therefore, the currently described RAS is nearly entirely available to the ruminant animal versus the 10-20% availability of conventional corn.

Corn breeding has increased vitreousness of corn and can comprise up to 60% of the starch-protein matrix. Increased Prolamin increases hydrophobic, alpha sulfur bonds and the gamma Zein bonds which are hydrophobic bonds in the protein of corn, thereby reducing the affinity to attach to rumen bacteria, fungi and protozoa for digestion.

It has been observed, and is presently disclosed that for a sample of RAS feedstuff manufactured according to the present teachings, and having an available starch content of 70.7% - - - when fed to the ruminant dairy cow, 37.3% of the starch was digested at 2 hours post-introduction to the rumen of the animal, 91.4% was digested at 7 hours, and 98.6% was digested at 24 hours.

The RAS feedstuff originating from corn and manufactured according to the present teachings is highly viscous causing attachment to rumen protozoa and bacteria which facilitates complete digestion. Furthermore, this RAS increases Microbial Bacteria (MB) and Microbial Protozoa (MP) thereby delivering higher levels of high quality protein to the small intestine and reducing the need for soluble protein sources. Still further, increased small intestine proteins reduce animal energy stress and increase pregnancy rates in animals under heat-stress, such as during the summer months of July, August and September. Surprisingly, dairy cows fed this RAS had a conception rate of 18% during the month of July while the control group that was not fed RAS experienced a 15.2 percent conception rate. Equally surprising, for August the finding was 18.7% versus 8.3% and for September the finding was 22.1% versus 12.5%.

Feeding RAS manufactured according to teachings of the present disclosure increases rumen pH thereby increasing fiber digesting bacteria (bugs) which enhance fiber digestion, protein synthesis and increased essential amino acid delivery to the small intestine. Also, RAS digestion kinetics increase attachment to endinomorphs (protozoa) which engulf themselves in starch, lyses and delivery high quality natural protein to the small intestine of the cow. Furthermore, the RAS feedstuff manufactured according to the present teachings increases rumen efficiency through increased protein and VFA delivery to the mammary gland and reducing DMI for greater kinetic efficiency.

In one example, a method is disclosed for processing a prolamin-containing feed source into a low-vitreous feedstuff and feeding the feedstuff to a ruminant animal for potentiating at least one of milk production and conception. The method comprises processing, by extrusion-type method, a prolamin-containing feed source comprising a starch-protein matrix wherein the included protein is composed of at least three percent prolamin and the result produces a hydrophilic, low-vitreous, gelatinous feedstuff having starch and protein content. The method comprises feeding the hydrophilic low-vitreous, gelatinous feedstuff to a ruminant animal, wherein a rumen-retained portion of the fed feedstuff is retained within the rumen of the animal for at least a twenty-four hour period, and during the first twenty-four hours of that period, at least seventy-five percent of the starch content of the rumen-retained portion of the fed feedstuff is digested.

In another embodiment, a method is disclosed for producing and feeding a feedstuff to a dairy cow and thereby potentiating milk production. The method comprises: heat-treating corn having a vitreousness of at least 66% for a period of at least 1200 seconds, the last 200 seconds of which is maintained above 90 degrees Celsius, thereafter extruding the corn and applying sufficient shear pressure to disrupt the prolamin/protein bonds and thereby obtaining an extruded hydrophilic, low-vitreous, gelatinous feedstuff comprising a starch and protein matrix composed of at least three percent prolamin. The feedstuff is then fed to a restrained dairy cow, thereby causing a portion of the feedstuff to be rumen-retained within the rumen of the animal for at least a twenty-four hour period such that during the first twenty-four hours of that period, at least seventy-five percent of the starch content of the rumen-retained portion of the fed feedstuff is digested.

In another embodiment, a method is disclosed for feeding an extruded feedstuff to a ruminant animal and thereby potentiating at least one of milk production and conception. The method comprises: obtaining an extruded, heat-treated corn having a vitreousness of at least 66% that has been heat-treated for a period of at least 1200 seconds, the last 200 seconds of which is maintained above 90 degrees Celsius and after which sufficient shear pressure has been applied by extrusion to disrupt the prolamin/protein bonds thereby producing an extruded hydrophilic, low-vitreous, gelatinous feedstuff comprising a starch and protein matrix composed of at least three percent prolamin; and feeding the feedstuff to a ruminant and thereby causing a portion of the feedstuff to be rumen-retained within the rumen of the animal for at least a twenty-four hour period such that during the first twenty-four hours of that period, at least seventy-five percent of the starch content of the rumen-retained portion of the fed feedstuff is digested.

In another embodiment, a method is disclosed for feeding an extruded feedstuff to a ruminant animal. The method comprises: feeding to a ruminant an extruded, hydrophilic, low-vitreous, gelatinous heat-treated feedstuff comprising corn having a vitreousness of at least 66% that has been heat-treated for a period of at least 1200 seconds, the last 200 seconds of which is maintained above 90 degrees Celsius and after which sufficient shear pressure has been applied by extrusion to disrupt the prolamin/protein bonds whereby the feedstuff comprises a starch and protein matrix composed of at least three percent prolamin, and thereby causing a portion of the feedstuff to be rumen-retained within the rumen of the animal for at least a twenty-four hour period such that during the first twenty-four hours of that period, at least seventy-five percent of the starch content of the rumen-retained portion of the fed feedstuff is digested.

In another embodiment, a method is disclosed for providing an extruded feedstuff to a ruminant animal. The method comprises: heat-treating corn for a period of at least 1200 seconds, the last 200 seconds of which is maintained above 90 degrees Celsius and thereafter extruding the corn and applying sufficient shear pressure to disrupt the prolamin/protein bonds and thereby obtaining an extruded hydrophilic, low-vitreous, gelatinous feedstuff; and feeding the extruded hydrophilic, low-vitreous, gelatinous feedstuff to a ruminant animal.

In at least one embodiment, a feedstuff for ruminant animals is disclosed. The feedstuff comprises: a composition that is at least 90% gelatinized, has a density of 18 to 24 lbs/ft³, a moisture content in the range of 5-10 percent and that contains at least 90% dissolved amylopectine.

In at least one embodiment, a feedstuff for ruminant animals is disclosed. The feedstuff comprises: a composition that is at least 60% gelatinized, has a density less than 30 lbs/ft³, a moisture content of less than 15 percent and which contains at least 70% dissolved amylopectine.

In at least one embodiment, a feedstuff for ruminant animals is disclosed. The feedstuff comprises: a composition that is at least 50, 60, 70, 80 or 90 percent gelatinized by heated and pressurized extrusion and that has a density of approximately 15, 20, 25 or 30 lbs/ft³ and is sufficiently light to float in ruminant fluid and has approximately 5, 10, 15 or 20 percent moisture content achieved by drying the composition and the composition contains at least 70, 80 or 90 percent dissolved amylopectine.

In another embodiment, a method is disclosed for producing a feedstuff for feeding to a dairy cow and thereby potentiating milk production. The method includes heat-treating corn for a period of at least 1200 seconds, of which 200 seconds is maintained above 90 degrees Celsius. Thereafter, the method includes extruding the corn and applying sufficient shear pressure to disrupt the prolamin/protein bonds and thereby obtaining an extruded hydrophilic, low-vitreous, gelatinous feedstuff comprising a starch and protein matrix composed of at least three percent prolamin. In this manner, the feedstuff, when fed to a dairy cow results in a portion of the feedstuff being rumen-retained within the rumen of the animal for at least a twenty-four hour period such that during the first twenty-four hours of that period, at least seventy-five percent of the starch content of the rumen-retained portion of the fed feedstuff is digested.

In another embodiment, a method is disclosed of producing a feedstuff for feeding to a dairy cow and thereby potentiating milk production. The method includes a multi-stage process comprising a first stage in which a feed corn for dairy cattle is heat-treated for a first period of time, of which at least 200 seconds is maintained above 90 degrees Celsius, and thereafter extruding the corn in a second stage by applying sufficient shear pressure to disrupt the prolamin/protein bonds. In this manner the method produces an extruded hydrophilic, low-vitreous, gelatinous feedstuff comprising a starch and protein matrix composed of at least three percent prolamin.

What is claimed is:

1. A method of producing a feedstuff, comprising:
heating a feed grain a first period of time at a temperature above 90 degrees Celsius; and
extruding the feed grain for a second period of time to produce a feedstuff comprising a starch and protein matrix comprising at least three percent prolamin.

2. The method of claim 1, wherein the first period of time is at least 100 seconds.

3. The method of claim 1, wherein the first period of time is at least 200 seconds.

4. The method of claim 1, wherein the temperature is at least 100 degrees Celsius.

5. The method of claim 1, wherein the temperature is at least 150 degrees Celsius.

6. The method of claim 1, wherein the temperature is at least 200 degrees Celsius.

7. The method of claim 1, wherein the starch and protein matrix comprises at least five percent prolamin.

8. The method of claim 1, wherein extruding comprises applying sufficient shear pressure to disrupt the prolamin/protein bonds and thereby producing an extruded hydrophilic, low-vitreous, gelatinous feedstuff.

9. The method of claim 1, wherein the feed grain comprises at least one of wheat, barley, rye, corn, sorghum and oats.

10. The method of claim 1, further comprising the feed grain having a vitreousness of at least 66% prior to the heating step.

11. The method of claim 1, wherein, the first period of time is at least 1200 seconds.

12. The method of claim 1, wherein, the feed grain is heated for at least a 200 second period during which the heat treatment is maintained above 90 degrees Celsius and the 200 second period is the last 200 seconds of the first time period of at least 1200 seconds.

13. A gelatinous feedstuff, comprising:
an extruded feed grain comprising starch and a protein matrix, the starch and protein matrix comprising at least three percent prolamin.

14. The feedstuff of claim 13, wherein the feed grain is selected from corn, wheat, barley, rye, sorghum, oats, and any combination thereof.

15. The feedstuff of claim 13, wherein the gelatinous feedstuff has a density of 30 lb/ft³ or less.

16. The feedstuff of claim 13, wherein the gelatinous feedstuff has a viscosity of less than 2,000 centipoise.

17. The feedstuff of claim 13, wherein the feedstuff comprises a starch content of at least eighty percent in a rumen-retained portion of fed feedstuff during a first twenty-four hours of an at least twenty-four hour period.

18. The feedstuff of claim 13, wherein the starch and protein matrix comprises at least five percent prolamin.
